(19) United States
(12) Patent Application Publication
(10) Pub. No.: US 2012/0219485 A1
(43) Pub. Date: Aug. 30, 2012

(54) ADDITIVE TO UREA SOLUTIONS

(75) Inventor: Sara Sebelius, Handen (SE)

(73) Assignee: ProppaBort AB, Katrineholm (SE)

(21) Appl. No.: 13/501,114

(22) PCT Filed: Oct. 1, 2010
(86) PCT No.: PCT/SE2010/051057

§ 371 Date: May 15, 2012

(30) Foreign Application Priority Data
Oct. 16, 2009 (SE) 0901329-3
Apr. 21, 2010 (SE) 1000404-2

(51) Int. Cl. B01D 53/90; C07C 43/13; C07C 43/11

(52) U.S. Cl. 423/235; 568/622; 568/678

(57) ABSTRACT

Use of compounds from the class of compounds Cₓ+₁H_{2X+3}(C₂H₄O)_{y}OH where x=(44y/W−44y+17/W−32)/14x≧0 1≦y≦20 0.58≦W≦1.0 as additive to urea solutions to counteract the risk of clogging of catalytic converters.

### TECHNOLOGICAL AREA

This invention concerns the use of compounds within the class of compounds Cₓ₊₁H₂ₓ₊₃(C₂H₄O)_{y}OH as additive to urea solutions for catalytic converters.

### The Standpoint of Technology

The predominant exhaust after-treatment technique for diesel engines on the market today is the Selective Catalytic Reduction (SCR). In SCR technology, the poisonous nitrogen oxides formed in the engine combustion are transformed into molecular nitrogen and water by reduction. The reducing agent, a 32.5 percent aqueous solution of urea that is kept in a tank connected to the catalytic converter, is injected before the catalytic converter and is subsequently split into ammonia in the catalytic converter. Ammonia reacts with the nitrogen oxides as follows:

NO_{X}+NH₃→N₂+H₂O

However, this technology is not problem-free. Instead of forming ammonia, urea may be transformed into various other products that form hard deposits and lumps which might clog or break the catalytic converter. On the market there are already additives designed to prevent the formation of lumps. These additives, however, are not mixable with the urea solution at room temperature, and therefore a preliminary mixing of the additive and the urea must be done in a factory. Furthermore, the additives currently on the market decompose into products that cause deposits which are difficult to dissolve. The additive in accordance with the invention is mixable with the urea solution at room temperature and does not decompose into products that may cause deposits.

### The Solution of the Problem

The invention at hand solves the problems of the known technique in accordance with the distinctive features stated in the following request for a patent.

### A DETAILED DESCRIPTION OF THE INVENTION

We have discovered that the class of compounds that we define below accelerates the transition from urea into ammonia, thus counteracting the risk of formation of lumps. Laboratory experiments have shown that the addition of only a small amount (<0.1%) of a compound out of this class of compounds to the urea solution nearly eliminates the risk of deposits and the formation of lumps in the catalytic converter. The compounds in this class of compounds are possible to mix with the urea solution at room temperature. This means that the premixing of the additive and the urea solution does not have to be done in a factory but that the additive can be poured directly into the tank where the urea solution is filled. The class of compounds has no negative impact on the environment and neither does it decompose into compounds that might damage the environment or the catalytic converters.

The application for a patent concerns the use of the following class of compounds as additive to urea solutions with the purpose of minimizing deposits and the formations of lumps in the SCR system:

Cₓ₊₁H₂ₓ₊₃(C₂H₄O)_{y}OH

where

x=|(44y/W−44y+17/W−32)/14|x≦0

1≦y≦20

0.58≦W≦1.0

### Experimental Part

Urea solution 1 was composed of a 32.5 percent aqueous solution of urea (the largest commercial available brand on the market).

Urea solution 2 was composed of a 32.5 percent aqueous solution of urea (another commercial available brand on the market).

Urea solution 3 was composed of the 32.5 percent aqueous solution of urea that is sold premixed with an additive (Diaxol®).

Urea solution 4 was composed of urea solution 1 with <0.1% of an additive of the class Cₓ₊₁H₂ₓ₊₃(C₂H₄O)_{y}OH. The mixing was done at room temperature.

Urea solution 5 was composed of urea solution 2 with <0.1% of an additive of the class Cₓ₊₁H₂ₓ₊₃(C₂H₄O)_{y}OH. The mixing was done at room temperature.

The urea solution was injected in a system of 250° C. and 350° C. (common exhaust temperatures for lump formation). All experiments were repeated twice.

### Experiment A

After sixteen hours at 250° C. white lumps the size of a decimeter had formed in both the experiments with urea solution 1 and urea solution 2, whereas the experiments with urea solution 3, urea solution 4 and urea solution 5 caused hardly any white deposits. With urea solution 3 insoluble black deposits had formed. Both inorganic and organic solvents were tested. No black deposits were observed for urea solution 4 and urea solution 5.

### Experiment B

After sixteen hours at 350° C. white lumps the size of a centimeter had formed in both the experiments with urea solution 1 and urea solution 2, whereas the experiments with urea solution 3, urea solution 4 and urea solution 5 caused no white deposits at all. With urea solution 3 insoluble black deposits had formed. The black deposits had begun to form as early as after half an hour. No black deposits were observed for urea solution 4 and urea solution 5.

The ability to reduce the formation of lumps of urea and its decomposition products (the white lumps) was consequently as good for urea solution 4 and urea solution 5 as for urea solution 3. Unique for urea solution 4 and urea solution 5 (urea solutions with additive Cₓ₊₁H₂ₓ₊₃(C₂H₄O)_{y}OH) was that they (unlike urea solution 3) caused no insoluble black deposits.

We observe the same effect for additives from the class of compounds Cₓ₊₁H₂ₓ₊₃(C₂H₄O)_{y}OH when y varies between 1 and 20. Neither C₃H₇(C₂H₄O)₁OH, C₆H₁₃(C₂H₄O)₄OH, C₉H₁₉(C₂H₄O)₁₀OH nor C₁₈H₃₇(C₂H₄O)₂₀OH caused any black deposits, and the white lumps of urea had nearly been eliminated in both experiment A and experiment B.

Comparative experiments have also been carried out with the previously known additives diethylene glycol and polyethylene glycol which show that they do not achieve as high an effect in reducing the formation of white lumps. See below.

When H(C₂H₄O)₂OH (diethylene glycol) and H(C₂H₄O)₁₀OH (polyethylene glycol) are used as additives, urea lumps are formed to a lesser extent than in the experiments without an additive (urea solution 1 and urea solution 2), but the ability to reduce the formation of lumps is not as high as the ability of the additives Cₓ₊₁H₂ₓ₊₃(C₂H₄O)_{y}OH. When diethylene glycol or polyethylene glycol is used as an additive to urea solution 1 and urea solution 2 and is injected during sixteen hours at 250° C. (compare experiment A) white urea lumps are formed that are a third of the size compared to the urea solutions without an additive. At an injection of sixteen hours at 350° C. (compare experiment B) white urea lumps are formed half the size compared to the urea solutions without an additive. The formation of lumps is accordingly reduced, but not at all to as high an extent as with additives from the class of compounds Cₓ₊₁H₂ₓ₊₃(C₂H₄O)_{y}OH.

We have also within our class of compounds been able to observe the importance of the length of the carbon chain of the additive. A shorter carbon chain gives an inferior effect to counteract the formation of urea lumps. CH₃(C₂H₄O)OH causes a little more white deposits than C₃H₇(C₂H₄O)OH. In the same way CH₃(C₂H₄O)₂OH shows somewhat inferior qualities to C₄H₉(C₂H₄O)₂OH, but both CH₃(C₂H₄O)₂OH and C₄H₉(C₂H₄O)₂OH show a better reducing ability toward the formation of white urea lumps than their corresponding ethylene glycols without a carbon chain (diethylene glycol).

1. (canceled)

2. An additive comprising a compound having the formula:
Cₓ₊₁H₂ₓ₊₃(C₂H₄O)_{y}OH
where
x=|(44y/W−44y+17/W−32)/14|x≧0
1≦y≦20
0.58≦W≦1.0
wherein a urea solution containing the additive minimizes deposits and the formation of lumps in a selective catalytic converter.

3. A method of minimizing deposits and the formation of lumps in a selective catalytic converter system for reducing nitrogen oxides in an exhaust gas stream, the method comprising:
adding to a urea solution a compound having the formula:
Cₓ₊₁H₂ₓ₊₃(C₂H₄O)_{y}OH
where
x=|(44y/W−44y+17/W−32)/14|x≧0
1≦y≦20
0.58≦W≦1.0; and then
injecting the urea solution into the exhaust gas stream upstream of the catalytic converter.
